(19) United States
(12) Patent Application Publication
(10) Pub. No.: US 2003/0040038 A1
(43) Pub. Date: Feb. 27, 2003

(54) INDUCIBLE REGULATORY SYSTEM AND USE THEREOF

(76) Inventors: STEVEN F. DOWDY, CLAYTON, MO (US); JOEL A. JESSEE, MOUNT AIRY, MD (US)

Correspondence Address: DIKE, BRONSTEIN, ROBERTS & CUSHMAN, Intellectual Property Practice Group, EDWARDS & ANGELL, P.O. Box 9169, Boston, MA, 02209, US

(21) Appl. No.: 09/134,793

(22) Filed: Aug. 14, 1998

Related U.S. Application Data
(60) Provisional application No. 60/056,713, filed on Aug. 22, 1997.

(51) Int. Cl. C12P 21/02; C12N 5/06; C12N 15/85

(52) U.S. Cl. 435/69.1; 435/455; 435/320.1; 435/325

(57) ABSTRACT

The present invention provides an inducible regulatory system in which transcription of a target nucleotide sequence in a host cell is activated by the introduction of a fusion protein having a transcription activator region and a protein transduction domain for entry of the fusion protein into the cell.

### CROSS-REFERENCE TO RELATED APPLICATION

The present application is a continuation of copending U.S. provisional application serial No. 60/056,713, filed Aug. 22, 1997, which is incorporated herein by reference.

### BACKGROUND OF THE INVENTION

1. Field of the Invention

The present invention relates to an inducible regulatory system in which transcription of a target nucleotide sequence in a host cell can be activated using a fusion protein having a transcription activator region and a protein transduction domain for entry of the fusion protein into the cell. The system can be used, for example, in a method of screening for the effect of a compound of interest on the host cell and in methods for activating transcription of DNA.

2. Background

Functional analysis of cellular proteins is greatly facilitated through changes in the expression level of the corresponding gene for subsequent analysis of the accompanying phenotype. For this approach, an inducible expression system controlled by an external stimulus is desirable.

Attempts to control gene activity have been made using various inducible promoters, such as those responsive to heavy metal ions, heat shock or hormones. However, these systems have not been completely successful because the inducer itself may evoke pleiotropic effects, which can complicate analyses. Additionally, many promoter systems exhibit high levels of basal activity in the non-induced state, which prevents shut-off of the regulated gene and results in modest induction.

An approach to circumventing these limitations is to introduce regulatory elements from evolutionary distant species such as E.coli into higher eukaryotic cells with the anticipation that effectors which modulate such regulatory circuits will be inert to eukaryotic cellular physiology and, consequently, will not elicit pleiotropic effects in eukaryotic cells. For example, the Lac repressor (lacR)/operator/inducer system of E.coli functions in eukaryotic cells and has been used to regulate gene expression. In one version of the Lac system, expression of lac operator-linked sequences is constitutively activated by a LacR-VP 16 fusion protein and is turned off in the presence of isopropyl-beta-D-thiogalactopyranoside (IPTG) (Labow et al. (1990) Mol. Cell. Biol., 10:3343-3356). The utility of these lac systems in eukaryotic cells is limited, in part because IPTG acts slowly and inefficiently in eukaryotic cells and must be used at concentrations which approach cytotoxic levels.

Components of the tetracycline (Tc) resistance system of E. coli have also been found to function in eukaryotic cells and have been used to regulate gene expression. For example, the Tet repressor (TetR), which binds to tet operator sequences in the absence of tetracycline and represses gene transcription, has been expressed in plant cells at sufficiently high concentrations to repress transcription from a promoter containing tet operator sequences (Gatz, C. et al. (1992) Plant J., 2:397-404). However, very high intracellular concentrations of TetR are necessary to keep gene expression down-regulated in cells, which may not be achievable in many situations, thus leading to “leakiness” in the system.

In other studies, TetR DNA binding domain (DBD) has been fused to a transactivation domain (TA) e.g., HSVI VP 16, to create a tetracycline-controlled transcriptional activator (tTA) (Gossen, M. and Bujard, H. (1992) Proc. Natl. Acad. Sci. USA, 89:5547-5551). The tTA, the DBD-TA protein, is kept at low levels of expression in the absence of tet. Upon the addition of tet, the DBD-TA dimerizes, binds stronger to the target DNA sequence contained not only in its own promoter, but also in the promoter of the cDNA to be induced. The DBD-TA induces itself (auto feedback) and this higher level of DBD-TA induces the target cDNA. In a doubly regulated system as this, the effect is a low level of transcription from the target cDNA until addition of tet.

This system has a number of drawbacks as well including, for example the following: (1) the constitutive expression of the DBD-TA fusion is toxic to the cells, (2) the DBD-TA fusion confers too high a basal level of transcription from itself and the target cDNA, in effect it is leaky, (3) the actual induction level of the target cDNA is not regulated, it can be very low or very high, (4) leaky expression of toxic or cell cycling arresting gene products in this system results in the inability to clone such transfected cells, (5) the system requires the transfection and stable integration of two plasmids, the DBD-TA containing plasmid and the target cDNA, (6) the system does not give linear expression on a single cell basis, that is cells from a “cloned” population can express 1×, 10×, or 100×levels of target cDNA product, and (7) transient transfection in normal or transformed cells can not be readily performed with this system.

Thus, there is a need for a more efficient inducible regulatory system which exhibits rapid and high level induction of gene expression and in which the inducer is tolerated by the host cells without cytotoxicity or pleiotropic effects.

### SUMMARY OF THE INVENTION

The present invention provides an inducible regulatory system in which transcription of a target nucleotide sequence in a host cell is activated by the introduction of a fusion protein having a transcription activator region and a protein transduction domain for entry of the fusion protein into the cell.

In one aspect of the present invention, the inducible regulatory system is used in a method of screening for the effect of a compound of interest (including nucleic acids such as cDNA) on a host cell by introducing into the cell a nucleotide sequence encoding the compound of interest operably linked to a regulatory sequence. A fusion protein comprising a protein transduction domain for entry of the fusion protein into the cell and a transcription activator region that binds to the regulatory sequence and activates transcription of the DNA is then introduced via transduction into the cell thus activating transcription of the DNA. The cell is then compared to a baseline control to determine the effect of the compound of interest on a target cell, e.g.. the resulting phenotype. For example, if the compound of interest is suspected of being a cell cycle arresting protein, the cDNA is transcribed and the effect of the expressed protein on the cell cycle can be determined.

The order in which the components of the fusion protein are linked is not important as long as each component can perform its intended function.

The baseline control may be the cell before introduction of the fusion protein, the cell in which the fusion protein has not been introduced, or the cell in which the fusion protein is non-functional, e.g.. has a non-functional transcription activator region.

The protein transduction domain of the fusion protein can be obtained from any protein or portion thereof that can assist in the entry of the fusion protein into the cell. Preferred proteins include, for example TAT, Antennapedia homeodomain and HSV VP22 as well as non-naturally occurring sequences. The suitably of a synthetic protein transduction domain can be readily assessed, e.g., by simply testing a fusion protein to determine if the synthetic protein transduction domain enables entry of the fusion protein into cells as desired.

The transcription activator region (TAR) of the fusion protein may be any protein or fragment that binds to the regulatory DNA sequence and activates transcription or transcribes the DNA. Such proteins include bacteriophage RNA polymerases, e.g., T7, SP6, GH1 and T3, and DNA binding proteins having gene activation function and possessing a DNA binding domain and a transactivation domain, e.g., E2F-1, C-Myb, Fos, Gal4, EST1 and Elf-1.

Chimeric proteins having a DNA binding domain from one protein and a transactivation domain from a different protein also may be used as the TAR. The TAR must however be compatible with the regulatory sequence, i.e., the TAR must be capable of binding to the regulatory sequence and activating transcription. For example, if the TAR is a bacteriophage RNA polymerase then the regulatory sequence is the promoter sequence that the RNA polymerase binds to. If the TAR is a chimeric protein having a DNA binding domain from Gal4 and a transactivation domain from cMyb, then the regulatory sequence includes at least the Gal4 enhancer element, which the DNA binding domain binds to, and a promoter region.

Preferred sources for obtaining the DNA binding domain include E2F-1, C-Myb, Fos, Gal4, ESTI, Elf-I and T7 RNA polymerase.

Preferred sources for obtaining the transactivation domain include E2F-1, cVilyb and VP16.

The fusion protein may also contain a nuclear localization signal.

The invention further provides a method for activating transcription of a target nucleotide sequence operably linked to a regulatory sequence in a host cell by introducing the fusion protein of the present invention into the cell.

In preferred methods of the invention, the fusion protein is introduced into the cell where at least a portion of the protein is denatured. It has been surprisingly found that rate and quantity of protein uptake into the cell is significantly enhanced relative to introduction of protein in a low energy folded confirmation.

The compound of interest can include, or the target nucleotide sequence encode, proteins, e.g., cytokines, tumor suppressors, antibodies, receptors, muteins, fragments or portions of such proteins, and active RNA molecules, e.g., an antisense RNA molecule or ribozyme.

The host cell may be a cell cultured in vitro or a cell present in vivo.

The invention also provides fusion proteins and nucleic acids encoding these proteins. In addition to the protein transduction domain and the transcription activator region, the fusion protein may contain other regions, e.g., a protein purification tag, or a protein identification tag such as MYC.

Further, fusion proteins of the invention can be expressed in insoluble form, particularly where the expressed fusion protein forms inside inclusion bodies. The protein then can be purified from the inclusion bodies by known procedures such as affinity chromatography. Expression of the fusion protein in insoluble form can be a significant advantage as it protects the expressed protein from degradation by host cell proteases, and thereby can substantially increase yields.

Other aspects of the invention are disclosed infra.

### BRIEF DESCRIPTION OF THE DRAWINGS

FIG. 1 is a plasmid map of pTAT/pTAT-HA.

FIG. 2 shows nucleotide and amino acid sequences of pTAT linker and pTAT HA linker.

### DETAILED DESCRIPTION OF THE INVENTION

In the inducible regulatory system of the invention, transcription of a target gene is activated by a transcription activator region of a fusion protein, also having a protein transduction domain for entry of the fusion protein into the cell. One aspect of the invention thus pertains to fusion proteins and nucleic acids (e.g., DNA) encoding fusion proteins. The term “fusion protein” is intended to describe at least two polypeptides, typically from different sources, which are operatively linked. With regard to the polypeptides, the term “operatively linked” is intended to mean that the two polypeptides are connected in manner such that each polypeptide can serve its intended function. Typically, the two polypeptides are covalently attached through peptide bonds. The fusion protein is preferably produced by standard recombinant DNA techniques. For example, a DNA molecule encoding the first polypeptide is ligated to another DNA molecule encoding the second polypeptide, and the resultant hybrid DNA molecule is expressed in a host cell to produce the fusion protein. The DNA molecules are ligated to each other in a 5′ to 3′ orientation such that, after ligation, the translational frame of the encoded polypeptides is not altered (i.e.,. the DNA molecules are ligated to each other in-frame).

The fusion protein of the invention is composed, in part, of a first polypeptide, referred to as the protein transduction domain, which provides for entry of the fusion protein into the cell. Peptides having the ability to provide entry of a coupled peptide into a cell are known in the art and include those obtained from TAT (Frankel, A. D., & Pabo, C. (1988), Cell, 55:1189-1193 and Fawell, S., et al., (1994) PNAS USA, 91:664-8.), Antennapedia homeodomain, referred to as “Penetratin” Ala-Lys-Ile-Trp-Phe-Gln-Asn-Arg-Arg-Met-Lys-Trp-Lys-Lys-Glu-Asn (SEQ ID. NO: 1) (Derossi et al., (1994) J. Bio. Chem., 269:10444-10450) and HSV VP22 (Elliot and O'Hare (1997) 88:223-234). The preferred protein transduction domain from TAT has the following amino acid sequence YGRKKRRQRRR (SEQ D. NO: 2). The protein transduction domain may be flanked by glycine residues to allow for free rotation.

The first polypeptide of the fusion protein is operatively linked to a second polypeptide, referred to as a transcription activator region (TAR), which binds to the regulatory sequence of the gene of interest and activates transcription or transcribes. To operatively link the first and second polypeptides, typically nucleotide sequences encoding the first and second polypeptides are ligated to each other in-frame to create a chimeric gene encoding a fusion protein.

Polypeptides which can function to activate transcription and can be used as the transcription activator region are well known in the art and include any protein or fragment that binds to the regulatory sequence and activates transcription of or transcribes the nucleotide sequence. Such proteins include bacteriophage RNA polymerases and DNA binding proteins having a gene activating function and possessing a DNA binding domain and a transactivation domain.

Bacteriophage RNA polymerases and their promoters include, for example, those obtained from the bacterial viruses T7 (Davanbo, P. et al., (1984) PNAS 81: 2035-2039), SP6 (Butler and Chamberlin (1982) J Biol. Chem., 257:5772-5778), GH1 and T3. The T7 RNA polymerase promoter can be obtained from pET-I Id (Studier et al., Enzymol. 185:60-89 (1990). For a further discussion on the specificity and individual promoters recognized by the bacteriophage RNA polymerases see Chamberlin et al, The Enzymes, 15:82-108 (1982); and Dunn et al, J. Mol. Biol., 166:477-535 (1983).

Preferred DNA binding proteins include E2F-1, C-Myb, Fos, Gal4, ESTI and Elf-1.

Chimeric TAR proteins having a DNA binding domain from one protein and a transactivation domain from a different protein may also be used. In such a situation it is not necessary that the DNA binding domain and the transactivation domain be adjacent in the fusion protein construct. The components of the fusion protein can be in any order as long as each is capable of performing its intended function. For example, the protein transduction domain can be flanked by the DNA binding domain and the transactivation domain.

The TAR must be compatible with the regulatory sequence, i.e., the TAR must be capable of binding to the regulatory sequence and activating transcription. For example, if the TAR is a bacteriophage RNA polymerase then the regulatory sequence would be the promoter sequence that the RNA polymerase binds to. If the TAR is a chimeric protein having a DNA binding domain from Gal4 and a transactivation domain from cMyb, then the regulatory sequence would include at least the Gal4 enhancer element and a promoter sequence.

Preferred sources for obtaining the DNA binding domain include E2F-I (AA 89-184), C-Myb (AA 34-189), Fos (AA 138-192), Gal4 (AA 1-38), EST1 AA 335-415) and Elf-I (AA 603-865).

Transcription activation domains of many DNA binding proteins have been described and have been shown to retain their activation function when the domain is transferred to a heterologous protein. A preferred polypeptide for use in the fusion protein of the invention is the herpes simplex virus vision protein 16 (referred to herein as VP16, the amino acid sequence of which is disclosed in Triezenberg, S. Jet al. (1988) Genes Dev. 2:718-729). At least one copy of about amino acids 411-490 from the C-terminal region of VP16 which retain transcriptional activation ability is used as the transactivation domain. Suitable C-terminal peptide portions of VP 16 are described in Seipel, K. et al. EMBO J., (1992) 13:4961-4968. Other preferred sources for obtaining the transactivation domain include E2F-1 (AA 368-437) and cMyb (AA 275-325).

Other polypeptides with transcriptional activation ability can be used in the fusion protein of the invention. Useful transcriptional activation domains, are disclosed in Seipel, K. et al., EMBO J., (1992) 13:4961-4968.

In addition to previously described transcriptional activation domains, novel transcriptional activation domains, which can be identified by standard techniques, are within the scope of the invention. The transcriptional activation ability of a polypeptide can be assayed by linking the polypeptide to another polypeptide having DNA binding activity and determining the amount of transcription of a target sequence that is stimulated by the fusion protein. For example, a standard assay used in the art utilizes a fusion protein of a putative transcriptional activation domain and a Gal4 DNA binding domain (e.g., amino acid residues 1-93). This fusion protein is then used to stimulate expression of a reporter gene linked to Gal4 binding sites (see e.g., Seipel, K. et al. (1992) EMBO J., 11:4961-4968 and references cited therein).

The regulatory sequence also includes a minimal promoter sequence which is not itself transcribed but which serves (at least in part) to position the transcriptional machinery for transcription. The minimal promoter sequence is linked to the transcribed sequence in a 5′ to 3′ direction by phosphodiester bonds (i.e., the promoter is located upstream of the transcribed sequence) to form a contiguous nucleotide sequence. The term “minimal promoter” is intended to describe a partial promoter sequence which defines the start site of transcription for the linked sequence to be transcribed but which by itself is not capable of initiating transcription. Thus, the activity of such a minimal promoter is dependent upon the binding of the transcription activator domain of the fusion protein of the invention to an operatively linked regulatory sequence. A minimal promoter can be obtained from the human cytomegalovirus (as described in Boshart et al. (1985) Cell, 41:521-530). Preferably, nucleotide positions between about +75 to −53 and +75 to −31 are used. Other suitable minimal promoters are known in the art or can be identified by standard techniques. For example, a functional promoter which activates transcription of a contiguously linked reporter gene (e.g., chloramphenicol acetyl transferase, beta-galactosidase or luciferase) can be progressively deleted until it no longer activates expression of the reporter gene alone but rather requires the presence of an additional regulatory sequence(s).

In a typical configuration, the enhancer element is operatively linked upstream (i.e., 5′) of the minimal promoter sequence through a phosphodiester bond at a suitable distance to allow for transcription of the target nucleotide sequence upon binding of the DNA binding domain of the fusion protein to the enhancer element.

In addition a fusion protein of the invention can contain an operatively linked to a third polypeptide which promotes transport of the fusion protein to a cell nucleus. Amino acid sequences which, when included in a protein, function to promote transport of the protein to the nucleus are known in the art and are termed nuclear localization signals (NLS). Nuclear localization signals typically are composed of a stretch of basic amino acids. When attached to a heterologous protein (e.g., a fusion protein of the invention), the nuclear localization signal promotes transport of the protein to a cell nucleus. The nuclear localization signal is attached to a heterologous protein such that it is exposed on the protein surface and does not interfere with the function of the protein. Preferably, the NLS is attached to one end of the protein, e.g. the N-terminus. The SV40 nuclear localization signal is a non-limiting example of an NLS that can be included in a fusion protein of the invention. The SV40 nuclear localization signal has the following amino acid sequence: Thr-Pro-Pro-Lys-Lys-Lys-Lys-Arg-Lys-Val (SEQ ID NO: 3). Preferably, a nucleic acid encoding the nuclear localization signal is spliced by standard recombinant DNA techniques in-frame to the nucleic acid encoding the fusion protein (e.g., at the 5′ end).

The fusion protein can also contain an operatively linked polypeptide such as a purification tag (which allows for purification of the protein) or an identification tag.

The DNA encoding the fusion protein can be inserted into an appropriate expression vector, i.e., a vector which contains the necessary elements for the transcription and translation of the inserted protein-coding sequence. A variety of host-vector systems may be utilized to express the protein-coding sequence. These include mammalian cell systems infected with virus (e.g., vaccinia virus, adenovirus, etc.); insect cell systems infected with virus (e.g., baculovirus); microorganisms such as yeast containing yeast vectors, or bacteria transformed with bacteriophage DNA, plasmid DNA or cosmid DNA. Depending on the host-vector system utilized, any one of a number of suitable transcription and translation elements may be used.

Once obtained, the fusion proteins can be separated and purified by appropriate combination of known techniques. These methods include, for example, methods utilizing solubility such as salt precipitation and solvent precipitation, methods utilizing the difference in molecular weight such as dialysis, ultra-filtration, gel-filtration, and SDS-polyacrylamide gel electrophoresis, methods utilizing a difference in electrical charge such as ion-exchange column chromatography, methods utilizing specific affinity such as affinity chromatograph, methods utilizing a difference in hydrophobicity such as reverse-phase high performance liquid chromatograph and methods utilizing a difference in isoelectric point, such as isoelectric focusing electrophoresis, metal affinity columns such as Ni-NTA.

As discussed above, fusion proteins of the invention can be expressed in insoluble forms. That can avoid proteolytic degradation of the fusion protein, significantly increase protein yields and increase delivery of fusion protein into target cells. The insoluble protein can be purified by known procedures such as affinity chromatography or other methods as detailed above.

Nucleic acid containing the target nucleotide sequence operably linked to a regulatory sequence can be introduced into a host cell transiently, or more typically, for long term regulation of gene expression, the nucleic acid is stably integrated into the genome of the host cell or remains as a stable episome in the host cell. For example, a recombinant expression vector is used to introduce the nucleic acid into the host cell.

As used herein, the term “host cell” is intended to include any cell or cell line, including prokaryotic and eukaryotic cells including, but not limited to, yeast, fly, worm, plant, frog, mammalian cells and organs. Non-limiting examples of mammalian cell lines which can be used include CHO dhfr- cells (Urlaub and Chasm (1980) Proc. Natl. Acad. Sci. USA, 77:4216-4220), 293 cells (Graham et al. (1977) J Gen. Virol., 36:59) or myeloma cells like SP2 or NSO (Galfre and Milstein (1981) Meth. Enzymol., 73(B):3-46).

In addition to cell lines, the invention is applicable to normal cells, such as cells to be modified for gene therapy purposes or embryonic cells modified to create a transgenic or homologous recombinant animal. Examples of cell types of particular interest for gene therapy purposes include hem atopoietic stem cells, myob lasts, hepatocytes, lymphocytes, neuronal cells and skin epithelium and airway epithelium. Additionally, for transgenic or homologous recombinant animals, embryonic stem cells and fertilized oocytes can be modified to contain nucleic acid encoding a target DNA. Moreover, plant cells can be modified to create transgenic plants.

Host cells encompass non-mammalian eukaryotic cells as well, including insect (e.g., Sp. frugiperda), yeast (e.g., S.cerevisiae, S. pombe, P. pastoris. K. lactis, H. polymorpha; as generally reviewed by Fleer, R. (1992) Current Opinion in Biotechnology, 3(5):486496)), fungal and plant cells.

Host cells encompasses prokaryotic cell as well, including E. coli and Bacillus.

Nucleic acid comprising the target nucleotide sequence operably linked to a regulatory sequence can be introduced into a host cell by standard techniques for transfecting cells. The term “transfecting” or “transfection” is intended to encompass all conventional techniques for introducing nucleic acid into host cells, including calcium phosphate co-precipitation, DEAE-dextran-mediated transfection, lipofection, electroporation, microinjection, viral transduction and/or integration. Suitable methods for transfecting host cells can be found in Sambrook et al. (Molecular Cloning: A Laboratory Manual, 2nd Edition, Cold Spring Harbor Laboratory press (1989)), and other laboratory textbooks.

The number of host cells transformed with the nucleic acid will depend, at least in part, upon the type of recombinant expression vector used and the type of transfection technique used. As aforesaid, nucleic acid can be introduced into a host cell transiently, or more typically, for long term regulation of gene expression, the nucleic acid is stably integrated into the genome of the host cell or remains as a stable episome in the host cell. Plasmid vectors introduced into mammalian cells are typically integrated into host cell DNA at only a low frequency. In order to identify these integrants, a gene that contains a selectable marker (e.g., drug resistance) is generally introduced into the host cells along with the nucleic acid of interest. Preferred selectable markers include those which confer resistance to certain drugs, such as G418 and hygromycin. Host cells transfected with the nucleic acid (e.g., a recombinant expression vector) and a gene for a selectable marker can be identified by selecting for cells using the selectable marker. For example, if the selectable marker encodes a gene conferring neomycin resistance, host cells which have taken up nucleic acid can be selected with G418. Cells that have incorporated the selectable marker gene will survive, while the other cells die.

Nucleic acid encoding the target nucleotide sequence operably linked to the regulatory sequence can be introduced into cells growing in culture in vitro by conventional transfection techniques (e.g., calcium phosphate precipitation, DEAE-dextran transfection, electroporation etc.). Nucleic acid can also be transferred into cells in vivo, for example by application of a delivery mechanism suitable for introduction of nucleic acid into cells in vivo, such as retroviral vectors (see e.g., Ferry, N. et al. (1991) Proc. Natl. Acad. Sci. USA, 88:8377-8381; and Kay, M. A. et al. (1992) Human Gene Therapy, 3:641-647), adenoviral vectors (see e.g., Rosenfeld, M. A. (1992) Cell, 68:143-155; and Herz, J. and Gerard, R D. (1993) Proc. Natl. Acad. Sci. USA, 90:2812-2816), receptor-mediated DNA uptake (see e.g., Wu, G. and Wu, C. H. (1988) J. Biol. Chem., 263:14621; Wilson et al. (1992) J Biol. Chem., 267:963-967; and U.S. Pat. No.5,166,320), direct injection of DNA (see e.g., Acsadi et al. (1991) Nature, 332:815-818; and Wolff et al. (1990) Science, 247:1465-1468) or particle bombardment (see e.g., Cheng, L. et al. (1993) Proc. Natl. Acad. Sci. USA, 90:4455-4459; and Zelenin, A. V. et al. (1993) FEBS Letters, 315:29-32). Thus, for gene therapy purposes, cells can be modified in vitro and administered to a subject or, alternatively, cells can be directly modified in vivo.

The host cells may be of a non-human transgenic organisms, including animals and plants, in which the nucleic acid encoding the target gene operably linked to a regulatory sequence is incorporated into one or more chromosomes in cells of the transgenic organism. Methods for generating transgenic animals, particularly animals such as mice, have become conventional in the art and are described, for example, in U.S. Pat. Nos. 4,736,866 and 4,870,009 and Hogan, B. et al., (1986) A Laboratory Manual, Cold Spring Harbor, N.Y., Cold Spring Harbor Laboratory.

The invention also provides a homologous recombinant non-human organism containing the target nucleotide sequence operably linked to the regulatory sequence. The term “homologous recombinant organism” as used herein is intended to describe an organism, e.g. animal or plant, containing a gene which has been modified by homologous recombination between the gene and a DNA molecule introduced into a cell of the animal, e.g.. an embryonic cell of the animal. In one embodiment, the non-human animal is a mouse, although the invention is not limited thereto. An animal can be created in which the target nucleotide sequence operably linked to the regulatory sequence has been introduced into a specific site of the genome, i.e., the nucleic acid has homologously recombined with an endogenous gene. Methods for creating a homologous recombinant plants and animals are known in the art.

In one embodiment, the target nucleotide sequence encodes a protein of interest. Thus, upon induction of transcription of the nucleotide sequence by the fusion protein and translation of the resultant mRNA, the protein of interest is produced in a host cell or animal. Alternatively, the nucleotide sequence to be transcribed can encode for an active RNA molecule, e.g., an antisense RNA molecule or ribozyme. Expression of active RNA molecules in a host cell or animal can be used to regulate functions within the host (e.g., prevent the production of a protein of interest by inhibiting translation of the mRNA encoding the protein).

A fusion protein of the invention can be used to regulate transcription of an exogenous nucleotide sequence introduced into the host cell or animal. An “exogenous” nucleotide sequence is a nucleotide sequence which is introduced into the host cell and typically is inserted into the genome of the host. The exogenous nucleotide sequence may not be present elsewhere in the genome of the host (e.g., a foreign nucleotide sequence) or may be an additional copy of a sequence which is present within the genome of the host but which is integrated at a different site in the genome. An exogenous nucleotide sequence to be transcribed and an operatively linked regulatory sequence can be contained within a single nucleic acid molecule which is introduced into the host cell or animal.

Alternatively, the present invention can be used to regulate transcription of an endogenous nucleotide sequence to which a regulatory sequence has been linked. An “endogenous” nucleotide sequence is a nucleotide sequence which is present within the genome of the host. An endogenous gene can be operatively linked to a regulatory sequence by homologous recombination between a regulatory sequence containing recombination vector and sequences of the endogenous gene using, for example, homologous recombination.

Another aspect of the invention pertains to kits which include the components of the inducible regulatory system of the invention. Such a kit can be used to regulate the expression of a target nucleotide sequence. In one embodiment, the kit includes a carrier means having in close confinement therein at least two container means: a first container means which contains a fusion protein of the invention, and a second container means which contains a recombinant vector for regulated transcription of a target nucleotide sequence. The vector comprises a nucleotide sequence linked by phosphodiester bonds comprising, in a 5′ to 3′ direction a first cloning site for introduction of a first nucleotide sequence to be transcribed, operatively linked to a regulatory sequence. The term “cloning site” is intended to encompass at least one restriction endonuclease site. Typically, multiple different restriction endonuclease sites (e.g., a polylinker) are contained within the nucleic acid.

To activate expression of a nucleotide sequence of interest using the components of the kit, the nucleotide sequence is cloned into the cloning site of the vector of the kit by conventional recombinant DNA techniques and then the vector is into a host cell or animal. The fusion protein is introduced into the host cell or animal to activate transcription of the nucleotide sequence of interest.

Another aspect of the invention pertains to methods for activating transcription of a nucleotide sequence operatively linked to a regulatory sequence in a host cell or animal. The methods involve introducing into the cell a fusion protein of the invention or administering a fusion protein of the invention to a subject containing the cell.

To induce gene expression in a cell in vitro, the cell is contacted with the fusion protein by culturing the cell in a medium containing the protein. When culturing cells in vitro in the presence of the fusion protein, a preferred concentration range for the fusion protein is between about 1 nM and about 1 mM. The fusion protein can be directly added to media in which cells are already being cultured.

To induce gene expression in vivo, cells within in a subject are contacted with the fusion protein by administering the fusion protein to the subject. The term “subject” is intended to include humans and other non-human mammals including monkeys, cows, goats, sheep, dogs, cats, rabbits, rats, mice, and transgenic and homologous recombinant species thereof. Furthermore, the term “subject” is intended to include plants, such as transgenic plants. When the fusion protein is administered to a human or animal subject, the dosage is adjusted to preferably achieve a serum concentration between about 1 nM and about 1 mM. The fusion protein can be administered to a subject by any means effective for achieving an in vivo concentration sufficient for gene induction. Examples of suitable modes of administration include oral administration (e.g., dissolving the inducing agent in the drinking water), slow release pellets, implantation of a diffusion pump and intravenous injection.

As discussed above, preferably a fusion protein is introduced into the cell where at least a portion of the protein is denatured. It has been surprisingly found that rate and quantity of protein uptake into the cell is significantly enhanced relative to introduction of protein in a low energy folded confirmation.

Denatured fusion protein for use in accordance with the invention can be produced by a variety of methods. For example, the fusion protein can be solubilized in urea, e.g. a 6-8 M urea solution, or other suitable agent, and loaded on a suitable column such Ni-NTA column (Qiagen) and washed with the urea solution. The fully denatured protein then can be refolded to a variety of conformations, e.g. by dialysis or Mono-Q or Mono-S chromatography on an FPLC (Pharmacia. Suitable dilaysis conditions include e.g. about 4° C. against 20 mM HEPES (pH 7.2)/150 mM NaCl. Suitable eluent for Mono-Q or Mono-S chromatography include use of an aqueous solution with an increasing salt concentration over time to elute the protein from the column, e.g. 50-500 mM NaCl. Such dialysis or chromatography will provide the fusion protein in a mixture of conformations, with only a minor portion in a lowest energy correctly refolded confirmation, e.g. about 25 percent of the protein may be in the low energy folded state. As referred to herein, a fusion protein that is at least partially denatured means that at least a portion of the protein sample (e.g. at least about 10, 15, 20, 30, 40, 50 60, 70 or 75 percent) of the protein is in a confirmation other than lowest energy refolded confirmation. As discussed above, such denatured fusion protein can be provided by treatment with a denaturing agent prior to contacting a cell with the protein.

The fusion protein in a mixture of conformations can then be transduced into desired cells, e.g. culturing the cells in the presence of the mixture such as by directly added the fusion protein to media in which the cells are being cultured as discussed above.

While not being bound by theory, it is believed that the higher energy denatured forms of a fusion protein of the invention are able to adopt lower energy conformations that can be more easily introduced into a cell of interest. In contrast, the protein in its favored folded confirmation will necessarily exist in a low energy state, and will be unable to adopt the relatively higher energy and hence unstable conformations that will be more easily introduced into a cell.

The invention is widely applicable to a variety of situations where it is desirable to be able to turn gene expression on and off, or regulate the level of gene expression, in a rapid, efficient and controlled manner without causing pleiotropic effects or cytotoxicity. Thus, the system of the invention has widespread applicability to the study of cellular development and differentiation in eukaryotic cells, plants and animals. For example, expression of oncogenes can be regulated in a controlled manner in cells to study their function.

By controlling gene expression, the present invention allows for the large scale production of a protein of interest. This can be accomplished using cultured cells in vitro which have been modified to contain a target nucleic acid encoding a protein of interest operatively linked to a regulatory sequence. For example, mammalian, yeast, fungal or bacterial cells can be modified to contain these nucleic acid components as described herein. The modified cells can then be cultured by standard fermentation techniques in the presence of the fusion protein to activate and control expression of the gene and produce the protein of interest.

The present invention further provides a production process for isolating a protein of interest. In the process, a host cell (e.g., a yeast, fungus or bacteria), into which has been introduced a nucleic acid encoding the protein of the interest operatively linked to a regulatory sequence, is grown at production scale in a culture medium in the presence of the fusion protein to stimulate transcription of the nucleotides sequence encoding the protein of interest and the protein of interest is isolated from harvested host cells or from the culture medium. Standard protein purification techniques can be used to isolate the protein of interest from the medium or from the harvested cells.

The system of the present invention can be used to keep gene expression “off” to thereby allow production of stable cell lines that otherwise may not be produced. For example, stable cell lines carrying genes that are cytotoxic to the cells can be difficult or impossible to create due to “leakiness” in the expression of the toxic genes. By repressing gene expression of such toxic genes using the present invention, stable cell lines carrying toxic genes may be created. Such stable cell lines can then be used to clone such toxic genes (e.g., inducing the expression of the toxic genes under controlled conditions using the fusion protein).

All documents mentioned herein are incorporated herein by reference.

The present invention is further illustrated by the following Examples. These Examples are provided to aid in the understanding of the invention and are not construed as a limitation thereof.

### EXAMPLE 1

Transcriptional Activation of a Target cDNA

The cell of interest is transfected with a DNA expression vector containing the regulatory DNA sequence followed by the open-frame of the cDNA/ gene of interest. The Green Flurorescent Protein (GFP) cDNA is placed downstream of the DNA regulatory sequence(s). GFP absorbs light near 488 r:n and emits near 530 nm thus allowing quantification of its expression (transcription) based on the intensity of the emission level on a device level on a device such as a flow cytometry sorter (FACS). Therefore, increased 530 nm light equals an increase in transcription of GFP.

1×10⁶ non-adherent Jurkat cells are transfected with 30 μg of the regulatory plasmid, washed in PBS(−) and allowed to recover for 6-24, or 48 hours. After the cells have recovered from the transfection process, purified fusion protein produced in bacteria, is added to the cell culture medium at concentrations from 1 nM, 10 nM, 100 nM, 1 μM, 10 μM, 100 μM. The fusion protein transduces across the cellular membrane and hence into the cell, then translocates to the nucleus by virtue of the NLS, binds the DNA regulatory sequence of the expression vector and activates transcription or transcribes the DNA.

A small aliquot, 1×10^{5,} of live Jurkat cells are then removed, placed in 200 μl of PBS(−) and analyzed on a FACS for detection of near 530 nm light emission. The cells are analyzed at 0, 3, 6, 12, 24, 36, 48 hours post transduction of the invention. The 530 nm light intensity level will increase proportionally as the GFP level increases. Low concentrations of the fusion protein will induce low levels of 530 nm light and as the concentration of fusion protein increases the 530 nm light intensity will increase. Positive and negative controls of no DNA regulatory sequence, the fusion protein minus one: PTD, DBD, TAR, and/or RNA polymerase.

### EXAMPLE 2

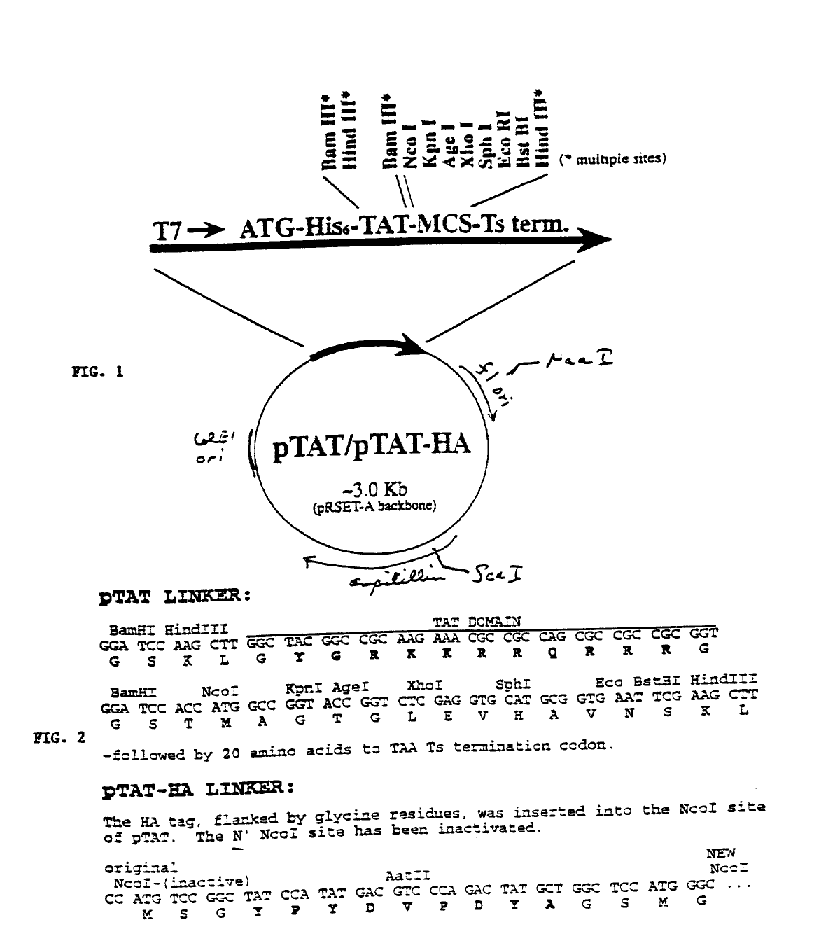

A preferred plasmid for TAT fusion protein expression was prepared as follows. A map of that plasmid is depicted in FIG. 1 of the drawings. FIG. 2 shows a nucleotide sequence (SEQ ID NO: 4) and amino acid sequence (SEQ ID NO: 5) of the pTAT linker as well as a nucleotide sequence (SEQ ID NO: 6) and amino acid sequence (SEQ ID NO: 7) of the pTAT-HA linker.

pTAT and pTAT-HA (tag) bacterial expression vectors were generated by inserting an oligonucleotide corresponding to the 11 amino acid TAT domain flanked by glycine residues to allow for free-bound rotation of the TAT domain (G-YGRKKRRQRRR-G) (SEQ ID NO: 8) into the Bam Hi site of pREST-A (Invitrogen). A polylinker was added C′ terminal to the TAT domain (see FIG. 1) by inserting a second oligonucleotide into the Nco I site (5′ or N′) and Eco RI site that contained NcoI-Kpnl-AgeI-XhoI-Sphl-EcoRI cloning sites. This is followed by the remaining original polylinker of the pREST-A plasmid that includes BstBI-Hind III sites.

The pTAT-HA plasmid was made by inserting an oligonucleotide encoding the HA tag (YPYDVPDYA SEQ ID NO: 3; see FIG. 2 where sequence is bold) flanked by glycines into the Ncol site of pTAT. The 5′ or N′ NcoI site was inactivated leaving only the 3′ or C′ to the HA tag followed by the above polylinker. The HA tag allows the detection of the fusion protein by immunob lot, immunoprecipitation or immunohistostaining by using 12CA5 anti-HA antibodies.

The nucleotide and amino acid sequences of each linker are set forth in FIG. 2. The pRSET-A backbone encodes ampicillin resistance, fl, ori, ColE1 ori (plasmid replication) and the transcript is driven by a T7 RNA polymerase promoter.

The invention has been described in detail with reference to preferred embodiments thereof. However, it will be appreciated that those skilled in the art, upon consideration of this disclosure, may make modifications and improvements within the spirit and scope of the invention.

What is claimed is:

1. A method of screening for the effect of a compound of interest on a target cell comprising:
a) introducing into the cell a DNA encoding the compound of interest operably linked to a regulatory sequence;
b) introducing into the cell a fusion protein comprising a protein transduction domain for entry of the fusion protein into the cell and a transcription activator region that binds to the regulatory sequence and activates transcription or transcribes the DNA;
c) comparing the cell to a baseline control.

2. The method of claim 1, wherein the baseline control is the cell before introduction of the fusion protein.

3. The method of claim 1, wherein the baseline control is a cell in which the fusion protein has not been introduced.

4. The method of claim 1, wherein the baseline control is a cell in which the fusion protein has a non-functional transcription activator region.

5. The method of claim 1, wherein the DNA regulatory sequence is obtained from a DNA sequence that is activated by E2F-1, cMyb 16 or Gal4.

6. The method of claim 1, wherein the protein transduction domain is obtained from a protein selected from TAT, Antennapedia homeodomain, HSV VP22 or a synthetic polypeptide.

7. The method of claim 1, wherein the transcription activator region comprises a DNA binding domain and a transactivation domain.

8. The method of claim 7, wherein the DNA binding domain and the transactivation domain are domains from a single protein.

9. The method of claim 7, wherein the DNA binding domain and the transactivation domain are domains from different proteins.

10. The method of claim 7, wherein the DNA binding domain is obtained from a protein selected from the group consisting of E2F-I, C-Myb, Fos, Gal4, EST 1 and Elf-1.

11. The method of claim 7, wherein the transactivation domain is obtained from a protein selected from the group consisting of E2F-1, cMyb and VP16.

12. The method of claim 1, wherein the transcription activator region comprises a bacteriophage RNA polymerase.

13. The method of claim 12, wherein the bacteriophage RNA polymerase is selected from the group consisting of T7, 5P6, GH1 and T3.

14. The method of claim 1, wherein the fusion protein further comprises a nuclear localization signal.

15. The method of claim 1 wherein the fusion protein is at least partially denatured when introduced into the cell.

16. A method for activating transcription of a DNA operably linked to a regulatory sequence in a host cell, comprising:
introducing into the cell a fusion protein comprising a protein transduction domain for entry of the fusion protein into the cell and a transcriptional activator that binds to the regulatory sequence and activates transcription of the target DNA.

17. The method of claim 16, wherein the regulatory sequence is obtained from a DNA sequence that is activated by E2F-1 or cMyb.

18. The method of claim 16, wherein the protein transduction domain is obtained from a protein selected from TAT, Antennapedia homeodomain, HSV VP22 or a synthetic polypeptide.

19. The method of claim 16, wherein the transcription activator region comprises a DNA binding domain and a transactivation domain.

20. The method of claim 19, wherein the DNA binding domain and the transactivation domain are each domains from a single protein.

21. The method of claim 19, wherein the DNA binding domain and the transactivation domain are domains from a different protein.

22. The method of claim 19, wherein the DNA binding domain is obtained from a protein selected from the group consisting of E2F-1, C-Myb, Fos, Gal4, EST1 and Elf-1.

23. The method of claim 19, wherein the transactivation domain is obtained from a protein selected from the group consisting of E2F-1, cMyb and VP16.

24. The method of claim 19, wherein the transcription activator region comprises a bacteriophage RNA polymerase.

25. The method of claim 24, wherein the bacteriophage RiNA polymerase is selected from the group consisting of T7, SP6, GH 1 and T3.

26. The method of claim 16, wherein the fusion protein further comprises a nuclear localization signal.

27. The method of claim 16 wherein the fusion protein is at least partially denatured when introduced into the cell.

28. A fusion protein comprising a protein transduction domain for entry of the fusion protein into the cell and a transcriptional activator that binds to the regulatory sequence and activates transcription or transcribes the target DNA.

29. The fusion protein of claim 28, further comprising a protein purification tag.

30. The fusion protein of claim 29, wherein the protein purification tag is a polyhistidine sequence.

31. The fusion protein of claim 28, wherein the fusion protein further comprises a nuclear localization signal.

33. The method of claim 32 wherein the expressed fusion protein forms inside inclusion bodies.

34. An isolated and purified DNA encoding the fusion protein of claim 28.

35. A plasmid that is pTAT/pTAT-HA.

36. A kit comprising:
a first container means which contains a recombinant vector for regulated transcription of a target nucleotide sequence, said vector comprising a nucleotide sequence linked by phosphodiester bonds comprising, in a 5′ to 3′ direction a cloning site for introduction of a nucleotide sequence to be transcribed, operatively linked to a regulatory sequence; and
a second container means which contains a fusion protein comprising a protein transduction domain for entry of the fusion protein into a cell and a transcriptional activator that binds to the regulatory sequence and activates transcription of the nucleotide sequence to be transcribed.


SEQUENCE LISTING (8 sequences)

SEQ ID NO: 1 - protein, 16 aa
AKIWFQNRRMKWKKEN

SEQ ID NO: 2 - protein, 11 aa
YGRKKRRQRRR

SEQ ID NO: 3 - protein, 10 aa
TPPKKKKRKV

SEQ ID NO: 4 - DNA, 105 bp; single-stranded, linear
ggatccaagcttggctacggccgcaagaaacgccgccagcgccgccgcggtggatccaccatggccggtaccggtctcgaggtgcatgcggtgaattcgaagctt

SEQ ID NO: 5 - protein, 35 aa
GSKLGYGRKKRRQRRRGGSTMAGTGLEVHAVNSKL

SEQ ID NO: 6 - DNA, 50 bp; single-stranded, linear
ccatgtccggctatccatatgacgtcccagactatgctggctccatgggc

SEQ ID NO: 7 - protein, 16 aa
MSGYPYDVPDYAGSMG

SEQ ID NO: 8 - protein, 13 aa
GYGRKKRRQRRRG